(19) United States
(12) Patent Application Publication
(10) Pub. No.: US 2018/0290386 A1
(43) Pub. Date: Oct. 11, 2018

(54) METHOD AND APPARATUS FOR SAFE AND ACCURATE 3D PRINTING

(71) Applicant: Vitae Industries, Inc., Providence, RI (US)

(72) Inventors: Daniel John DeCiccio, Winter Park, FL (US); Jeanine Sinanan-Singh, Orlando, FL (US); Francisco Schunk, Providence, RI (US)

(21) Appl. No.: 15/887,804

(22) Filed: Feb. 2, 2018

Related U.S. Application Data
(63) Continuation-in-part of application No. 15/330,809, filed on Nov. 7, 2016.
(60) Provisional application No. 62/454,286, filed on Feb. 3, 2017.
(60) Provisional application No. 62/251,318, filed on Nov. 5, 2015.

(51) Int. Cl. B29C 64/393; B29C 64/364; A61L 2/10; A61L 2/22; A61L 2/24

(52) U.S. Cl. CPC B29C 64/393; B29C 64/364; B29L 2031/753; A61L 2/22; A61L 2/24; A61L 2/10

(57) ABSTRACT

A method includes steps for creating safe and accurate pharmaceutical products with a 3D printer. A 3D printer is set up and a set of printer instructions are created, the instructions being designed to print a desired final product having at least one desired attribute. A product is printed based on the instructions. At least one attribute of the product is measured and compared with the desired attributes of the desired final product. If there is a difference between the product attributes and desired attributes, changes are made to the instruction set and a new product is printed. When there is a match between product attributes and desired attributes, a safe and accurate medical product has been created.

### CROSS REFERENCE TO RELATED APPLICATIONS

This application claims benefit from U.S. Provisional Patent Application No. 62/454,286, filed Feb. 3, 2017, and is a Continuation-in-Part of U.S. patent application Ser. No. 15/330,809 filed Nov. 7, 2016, which claims priority to U.S. Provisional Patent Application No. 62/251,318, filed Nov. 5, 2015, all of which are incorporated herein by reference.

### STATEMENT REGARDING GOVERNMENT INTEREST

None.

### BACKGROUND OF THE INVENTION

### 1. Field of the Invention

The subject disclosure relates to methods and systems for 3D printing, and more particularly to improved methods and systems for 3D printing that are self-contained, clean safe and/or capable of maintaining best manufacturing processes.

### 2. Background of the Related Art

Additive manufacturing, more commonly known as 3D printing, has become an increasingly popular method for the rapid prototyping and fabrication of a wide variety of products. 3D printers are now widely available to the general public due to recent low-cost models. One area of increased interest in 3D printing is the medical field where rapid and custom production can greatly benefit patients. Recently, the first 3D printed drug, Spritam levetiracetam, was FDA approved. 3D printed implants have been used in a wide variety of application from stents to cartilage replacement.

When producing medical products, maintaining a sterile working environment is key to eliminating contamination and infection. 3D printed implants must be sterilized after production using common techniques such as autoclaves, ethylene oxide chambers, solvent washes etc. 3D printed drugs should be produced in a sterile room or sterile environment to minimize contamination. The need for a clean, safe cGMP working environment is a limitation which prevents use of 3D printed bioproducts outside of facilities already designed to handle such sterile working conditions. For example, a rural clinic or small medical practice may not be able to safely 3D print urgent medical products like custom drugs, compounds or custom implants including teeth and emergency stents.

Although some 3D printers may be used under a vacuum hood for filtration of fumes and or particulate contaminates created during the printing processes, current models fail to create and maintain a highly clean environment for manufacturing.

Further, since 3D printers are used to create important medical products, accuracy is crucial. If a 3D printer is not producing medical products with a high degree of accuracy, it can be very hard to receive FDA approval, and more importantly, there is a risk that the health of a patient can be adversely impacted.

### SUMMARY OF THE INVENTION

The subject technology is directed to a method and apparatus for creating medical products and compounds via 3D printing in a reliably clean or sterile environment regardless of the presence of cGMP and/or sterile facility. Further the subject technology provides a 3D printer which can ensure that the products it creates are accurate.

In one embodiment, the subject technology is directed to an instrument for producing clean and/or sterile products. The instrument includes a housing defining a clean interior. The housing has an inlet and a first HEPA filter for filtering an inflow through the inlet to create filtered air. A plurality of UV-C lights irradiate the interior. A 3D printer is in the interior to produce clean products. Preferably, the interior is CGMP compliant. The instrument may also have micro spray nozzles for spraying a sterilizing agent within the interior. A pressure pump can create a pressure differential, positive or negative, within the housing. Typically, the housing has an outlet with a second HEPA filter for filtering an outflow through the outlet to create environmentally safe air. An air circulator can create a air flow through the housing.

The instrument may also have a sterile internal needle having an extrusion tip and a septum, wherein: the extrusion tip is operable to puncture the septum; and the sterile internal needle is operable to draw at least one sterile working material into the sterile interior. A plurality of replacement extrusion tips can be stored in the interior or in a similarly convenient location. The 3D printer includes sterile components. Further, the sterile interior, the HEPA filters, and the plurality of UV-C lights may be selectively coated with a sterilizable material. The sterilizable material is selected from the group consisting of: ceramic; metals; and polymers. Aluminum, polycarbonate, polyacrylamide, polyacrylic and the like are possible sterilizable materials.

Another embodiment of the subject technology is directed to a method for creating sterile pharmaceutical compounds from at least one component including the steps of: creating a work station by enclosing a 3D printer within an interior of a housing; filtering air for filling the interior; flooding the interior station with UV-C light; and producing a pharmaceutical compound within the work station using the 3D printer. The interior of the housing and 3D printer may be sterilized by the UV-C light. To further sterilize the work station, the method may mist a sterilizing agent over the interior of the work station. Filtered air is preferably circulated within the interior in a laminar pattern. The method may also include sterilizing and curing at least one component of the product by using one of the following: heat; or UV-C light.

The method may also include the steps of: forming a septum in a portion of the work area; puncturing the septum with a sterile internal needle, the sterile internal needle having a sterile extrusion tip; and drawing material into the working area using the sterile internal needle. The method also can include the steps of: storing at least one sterilized replacement tip in the work area; and after drawing material into the working area using the sterile internal needle, replacing the sterile extrusion tip with one of the sterilized replacement tips.

Still another embodiment of the subject technology is directed to an instrument for producing sterile or clean, safe non-sterile products including an inner chamber having a 3D printer, a pressure pump, a sterile internal needle, a septum, an air circulator, and a micro spray nozzle connected to a spray pump. A first outer chamber fluidly connects to the inner chamber. The first outer chamber includes: an inlet, a HEPA filter, and a UV-C light. A second outer chamber fluidly connects to an inner chamber. The second outer chamber includes: an outlet, a HEPA filter, and a UV-C light. One or more UV-C lights irradiate at least the inner chamber. Preferably, air within the inner chamber flows in a laminar pattern between the first outer chamber and the second outer chamber, and the pressure pump maintains a positive pressure within the inner chamber.

In at least one embodiment, the subject technology includes a method of creating safe and accurate medical products with a 3D printer. The method includes setting up a 3D printer and creating a set of printer instructions designed to print a desired final product. At least one product attribute is measured and compared with at least one desired attribute. A determination is made as to whether there is a difference between the product attribute(s) and the desired attribute(s). The printer instructions are modified if there is any difference, and a new product is printed based on the new printer instructions such that the new product attribute(s) can be compared to the desired attribute(s). When at least one product attribute matches at least one desired attribute, an ideal instruction set for creating safe and accurate products has been created. The ideal instruction set can be stored for later use. Future safe and accurate products can then be printed based on the ideal instruction set.

In at least one embodiment, one of the desired product attributes is a desired product weight and one of the product attributes is a first product weight. In this embodiment, the first product weight can be determined by weighing the product, for example, on a print stage configured for weight tracking. Additionally the print stage may include force sensing resistors and/or manometers. The desired product attributes can be selected depending on a medically recommended dose or a medically recommended volume. In at least some embodiments, the product attributes can be measured by laser based temperature measurements, spectroscopic measurements, or optical measurements. Further, in some embodiments, the method is carried out on a 3D printer in a clean or sterile environment.

It should be appreciated that the subject technology can be implemented and utilized in numerous ways, including without limitation as a process, an apparatus, a system, a device, a method for applications now known and later developed or a computer readable medium. These and other unique features of the system disclosed herein will become more readily apparent from the following description and the accompanying drawings.

### BRIEF DESCRIPTION OF THE DRAWINGS

So that those having ordinary skill in the art to which the disclosed system appertains will more readily understand how to make and use the same, reference may be had to the following drawings.

FIG. 1 is a front view of an instrument for producing and safe products in accordance with the subject disclosure.

FIG. 2 is an exploded view of the instrument of FIG. 1.

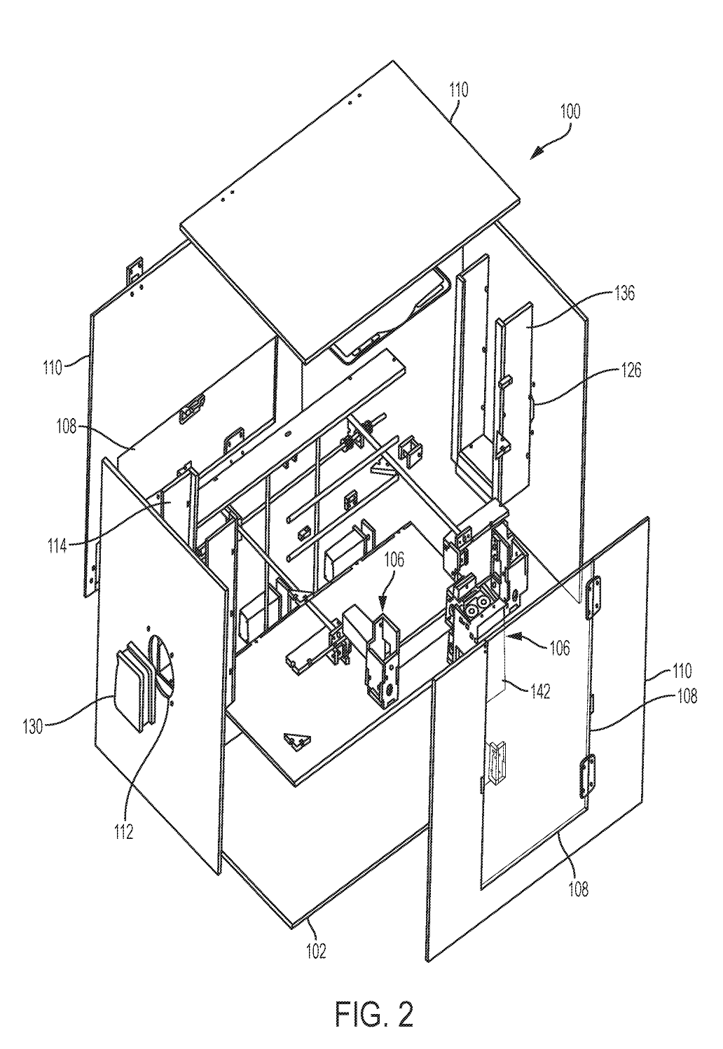

FIG. 3 is a flowchart illustrating a method for creating safe and accurate medical products with a 3D printer in accordance with the subject disclosure.

### DETAILED DESCRIPTION

The subject technology overcomes many of the prior art problems associated with producing sterile items fabricated using 3D printing. The advantages, and other features of the systems and method disclosed herein, will become more readily apparent to those having ordinary skill in the art from the following detailed description of certain preferred embodiments taken in conjunction with the drawings which set forth representative embodiments of the present invention and wherein like reference numerals identify similar structural elements.

In brief overview, the 3D printer of the subject disclosure is a fully self-contained unit, which allows sterile production of medical products without any additional outside matter. The 3D printer makes printing adjustments based on the differences between previously printed products and an ideal product. Primary ways in which a sterile internal environment/working area is maintained and printing adjustments are made are described in more detail below. The attached Figures and following description provide illustration of embodiments of the subject technology.

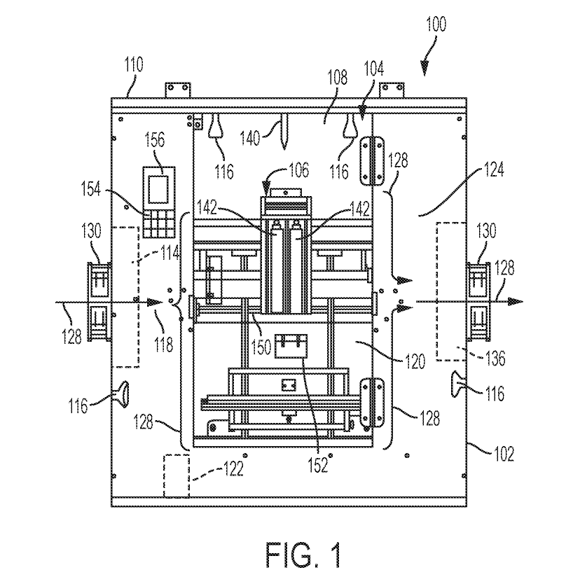

Referring now to FIG. 1, an instrument for producing sterile products in accordance with the subject disclosure is shown generally at 100. The instrument 100 includes a housing 102 which defines a sterile interior 104. The sterile interior 104 is used, in whole or in part, as a work station for creating the sterile product. To that end, a 3D printer 106 is included within the sterile interior 104 and is operable to produce sterile products. The housing 102 has one or more windows 108 for visual access to the interior 104. The windows 108 and panels 110 of the housing 102 may be hingedly connected and/or permanently fixed together.

The instrument 100 also includes an inlet 112 which draws air through a first high efficiency particulate arrestance (hereinafter HEPA) filter assembly 114, creating filtered air (not distinctly shown). Additional HEPA filters can be added to provide greater filtration or to increase the length of time before it becomes necessary to replace the filters. Additionally, UV-C lights 116 irradiate the sterile interior 104 of the instrument 100. The UV-C lights 116 improve sterilization by destroying and/or sterilizing pathogens such as bacteria, viruses, mold spores, cysts and the like. By irradiating the sterile interior 104, the UV-C lights 116 sterilize the air within the sterile interior 104 as well as the equipment within the sterile interior 104 such as the 3D printer 106.

In the embodiment shown, air entering through the inlet 112 is filtered through a HEPA filter assembly 114 and irradiated by UV-C lights 116 in a first outer area or chamber 118. Only after the air is filtered and irradiated in the first outer chamber 118 is the filtered air then circulated into an inner area or chamber 120 that includes the 3D printer 106. In other words, the external air is filtered and sanitized before introduction to the inner chamber 120 of the system 100. In this way, the inner chamber 120 can be used as a substantially sterile work station.

Further, an air circulator 122 causes the filtered and sterilized air to continuously move within the system 100 to minimize the chance of contamination while the unit is both idle and operating. For example, the air circulator 122 can move the air from top to bottom or side to side. Preferably, a laminar pattern moves the filtered air from the first outer chamber 118 to the inner chamber 120 to a second outer area or chamber 124 for egress from an outlet 126 as shown by flow arrows 128. Fans 130, mounted on the inlet 112 and outlet 126 respectively, help establish air flow. A second HEPA filter assembly 136 is also mounted on the outlet 126.

By continually moving air throughout the instrument 100, the air circulator 122 and fans 130 limit the potential for unfiltered air to enter the inner chamber 120. Further, by recirculating air within the system 100, such as sending filtered and sanitized air from the second outer chamber 124 back into the first outer chamber 118, air inside the system 100 can also be recirculated to reduce sanitization needs. The inner chamber 120 is also equipped with one or more pressure pumps 134 which can maintain a positive or negative pressure in the sterile interior 104 as desired. A positive pressure differential can further limiting the potential for unfiltered outside air to enter. A negative pressure differential can further limit outgas and the like from the interior. Alternatively, the system 100 can be sealed.

Air within the inner chamber 120 may, in some cases, be contaminated by the 3D printing process. Therefore, it may be desirable to filter and/or sterilize contaminated air within the system 100, either before the air is recirculated into the system 100, or before the air is reintroduced into the outside environment. Therefore, the second outer chamber 124 includes the second HEPA filter assembly 136 and UV-C light 116, for filtering and sterilizing air within the second outer chamber 124. In this way, the instrument can create filtered, sterilized air that is safe for recirculating within the instrument. It is envisioned that the outer chambers 118, 124 can be arranged in a variety of configurations depending upon the application. Additionally, the HEPA filter assemblies 114, 136 remove noxious fumes to create safe air for reintroduction into the outside environment via the outlet 126.

In one embodiment, the UV-C lights 116 are mounted over and under the working area to guarantee all surfaces can be sufficiently irradiated. In effect, the UV-C light guarantees there are no live pathogens on any of the working surfaces before fabrication. The UV-C lights 116 can also be used to cure/set UV-sensitive polymers and other materials as part of the 3D printing process. UV-C lights are commonly used and widely accepted/certified for medical sanitation of surfaces as well as water/air purification. The ability to sterilize the unit before construction allows a user to open the window 108 of the system 100, remove parts, perform maintenance and the like, then have the interior 104 sterilized and ready to use quickly.

The system 100 also includes a micro spray nozzle 140 for spraying a sterilizing agent within the sterile interior 104. The sterilizing agent can be hydrogen peroxide as an alternative or second level of protection for initial sterilization of the interior 104. A mist of sterilizing agent may be automatically sprayed over all surfaces from centrally located nozzles. After a short amount of time (e.g., less than 60 minutes), all surfaces would be sterile.

Air filtration and direction is another aspect addressed by the subject technology. HEPA (high efficiency particulate arrestance) filters which can be combined with additional UV-C lights supply clean air to the system and sterilize surfaces. In other words, the external air is filtered and sanitized before introduction to the work area of the system 100. Already sterile air from inside the system 100 can also be recirculated to reduce sanitization needs. The filtered and sterilized air continuously moves within the system to minimize the chance of contamination while the unit is both idle and operating. Filtration also serves to remove any noxious fumes and contain such fumes in the system.

In one embodiment, the system 100 has an air-tight seal such as a tightly fitted housing 102 to prevent external contamination from entering. In another embodiment, an air-tight seal from the outside world is not practical. Positive pressure is maintained within in the system 100 to limit the possibility of contaminants entering the system 100. The system 100 maintains a slight higher pressure within to prevent contamination from entering. Both positive pressure and laminar flow are currently used in clean rooms and some operating rooms to ensure sterile working areas. The 3D printer of the subject technology has the positive pressure and controlled flow built in so that parts are produced in a sterile environment.

The system 100 carefully introduces the sterile working materials. The materials the 3D printer uses to manufacture products are sterile to help the resulting product produced to be sterile. For example, the 3D printer 106 may use cartridges 142 that contain sterile ingredients for the resulting products. In a drug compounding application, the cartridges are pre-filled and FDA approved so that more common local pharmacies can utilize the subject technology to produce drug compounds. Another application is cartridges with medical cannabis for approved dispensaries to create a variety of 3D printed products on site. The subject technology is significantly more advantageous as by producing the product in a clean or sterile environment with sterile cartridges, subsequent sterilization is not needed.

Contaminated components may be sterilized by the manufacturing process due to application of heat (e.g., heat lamps), the UV light, the process needed to extrude or cure the products, and/or the sterile misting and the like. In the case of heated extruded material, if the material is not already sterile, it would need to be heated to appropriate temperatures during extrusion for sterility. The 3D printer may include a heating assembly or the heat could be delivered from a secondary source such as warmed controlled air flow. The controlled air flow assists with carrying away particulate contaminants created during the heating/extrusion process so that the particulate contaminants are not incorporated in the product. Unsterile working materials will be held in a separate area of the devices which is sealed off to prevent contamination of sterile environments.

Sterile working materials can also be efficiently incorporated into the work area. In one embodiment, the sterile working material arrives. The working material is drawn into the working area by a sterile internal needle puncturing a septa or dividing wall and drawing the clean material into the sterile working area. The tools/tips/needs (e.g., needle) and septa can be sterilizable through techniques including but not limited to the aforementioned techniques or replaceable. In the case of disposable tips, sterile extrusion tips (such as syringe or micropipette tips which are shipped sterile) will be stored within the system's clean and sterilized area. After one tip is finished, the 3D printer can automatically dispose of the used tip and insert a new tip as needed. Preferably, materials within the working area are made of or coated with a material which can be readily sterilized such as ceramic, metals or certain polymers. Additional sterilization techniques such as autoclave or like functionality where heat and pressure ensure sterilization can also be employed to sterilize the working area and components before and after use.

It is envisioned that the subject technology results in a work environment that is Current Good Manufacturing Practice (CGMP) regulations compliant as set forth by the US Food and Drug Administration (FDA). As CGMP are modified, the subject technology can be adapted to comply with cGMP.

As can be seen from review of the above, built in, self-contained sterilization allows 3D printers to be used in almost any environment and still produce medically usable products. In the case of 3D printed drugs, even a small business or pharmacy could produce pharmaceutical quality products. Pharmaceutical companies do not need to produce products in clean rooms because the clean room is built in to the 3D printer allowing the same cleanliness standards to be achieved. For the production of implants, surgeons and patients would be able to know a product that is produced on demand is as sterile as any traditionally produced and sterilized implant.

Referring to FIG. 1, the 3D printer also includes a printer stage 150. The 3D printer creates a printed product by discharging and arranging the contents of the cartridge 142 directly onto the printer stage 150. The printer stage 150 may also be equipped with a variety of components, not shown herein, for monitoring the products which are created. For example, the print stage may be equipped with force sensing resistors, manometers, or other devices for measuring the force or weight applied to the printer stage 150 by a product. Alternatively, or additionally, the printer stage 150 may be furnished with equipment capable of determining a number of characteristics of a product on the printer stage 150, such as weight, volume, or shape, for example. For example, the printer stage 150 may include apparatus for taking laser based temperature measurements, spectroscopic measurements, or optical measurements.

The 3D printer may also be connected to a controller 152 which is capable of a variety of specialized tasks. For example, the controller 152 may be one or more application specific integrated circuits. The controller 152 receives input from the 3D printer 106, the printer stage 150, and the various components which are part of the printer stage 150 as described above. A user can input data or instructions into the controller 152 via a keypad 154 and can view data output from the controller 152 in display monitor 156. In alternative embodiments, instead of the keypad 154 and display monitor 156, other standard input/output devices may be connected to the controller 152. The controller may also include Wi-Fi capability to communicate with other remote servers.

In some embodiments, the controller 152 may have processing capability and a memory. For example the controller 152 may include a processor which is generally logic circuitry that responds to and processes instructions. The processor can include, without limitation, a central processing unit, an arithmetic logic unit, a number of application specific integrated circuits, a task engine, and/or any combinations, arrangements, or multiples thereof. The processor is in communication with memory. Typical memory includes random access memory (RAM), rewritable flash memory, read only memory (ROM), mechanisms and structures for performing I/O operations, and a storage medium such as a magnetic hard disk drive(s). The memory may be a combination of integral and external memory.

The memory includes software and a plurality of modules as needed to perform the functions of the subject technology. A module is a functional aspect which may include software and/or hardware. Typically, a module encompasses the necessary components to accomplish a task. It is envisioned that the same hardware (e.g., memory and processor) could implement a plurality of modules and portions of such hardware being available as needed to accomplish the task. For example, a database module creates, stores, and maintains data and multiple databases necessary for the proper operation of some facets of the subject technology. A program module stores an instruction set to allow the operator to program operation of the printer. An algorithm module stores an instruction set to allow the processor to apply one or more algorithms to operation of the printer as well as vary the actual algorithms according to user input.

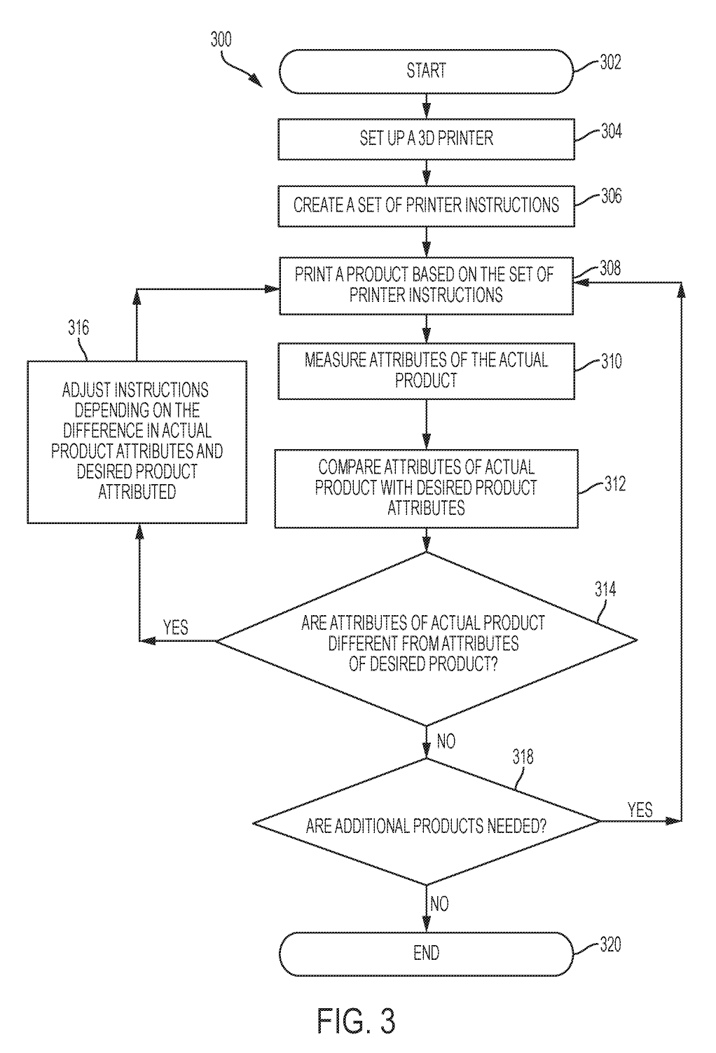

Referring now to FIG. 3, a flowchart 300 for a method using a 3D printer to create one or more safe and accurate medical products is shown. The method 300 can be used for assembling a variety of medical products such as a 3D printed drug or implant. The method starts at step 302, and at step 304 a 3D printer is set up and prepared for printing. In at least one embodiment the 3D printer is set up such that it is contained in a completely sterile environment, as described above.

Next, at step 306, the user creates a set of printer instructions which will be implemented by the 3D printer. The instructions include a series of commands which allow the 3D printer to create a desired product having one or more desired attributes. For example, if a user desired to create a pill, such as a pill of 50 mg of acetaminophen, the instructions can be designed to print a product which is has the general shape of a prolate spheroid and a mass of approximately 50 mg. The instructions can be input into the controller 152 by the user, or incorporated into the controller by an instructions module to be carried out automatically. At step 308, a product is 3D printed onto the print stage 150 based on the instructions.

Once the product is created, the product rests on the print stage 150. On the print stage 150, the product can be subjected to the measuring components contained on or around the print stage 150, as described above. Therefore at step 310 various attributes of the product on the print stage 150 are measured. For example, in some embodiments, the products weight, volume, and/or shape are measured. Other attributes can also be measured in accordance within the capabilities of the measuring components, as described above. The measured attributes of the product are then compared to the attributes of a desired product at step 312. This comparison can be done manually by a user reviewing output from the controller, or automatically by an analysis module designed to compare the product characteristics to desired characteristics. Once the attributes have been compared, the user, or analysis module, will determine whether the designed characteristics are significantly different from the product characteristics at step 314. If the product characters are significantly different than the desired characteristics the user proceeds to step 316 to modify the instructions. Whether the difference is significant will depend largely on the type of product. For example, a difference in volume of 5% might not be significant for a tablet of acetaminophen, especially if the tablet has the correct mass. However, for a spinal implant a difference in volume of 5% between the volume of the product and the desired volume may cause a user discomfort or even more serious long term medical problems. Therefore for various applications, a user can determine the difference between an actual product and a desired product that should be considered significant for each measured characteristic. If the method is being carried out automatically, the amount of difference considered significant can be set within the analysis module. In any case, desired product attributes and significant differences can be selected depending on a medically recommended dose, medically recommended volume, or other medical guidelines determined by a medical professional.

At step 316, the instructions are modified, based on the differences in actual attributes and desired attributes, such that the new instruction set is expected to create a product more akin to the desired product. In way of simple example, if the user desired to create a 50 mg tablet of acetaminophen, and the tablet that was actually printed ended up having a mass of 55 mg, the user could revise the instructions such that the 3D printer prints approximately 10 percent less the next time it prints a product. One skilled in the art would understand that other modifications of a similar vein can be made to adjust the printing instructions such that the attributes of the final product are more similar to the attributes of the desired product.

With respect to steps 314 and 316, in at least some embodiments, it will be beneficial for a user to also consider past differences between actual product attributes and desired product attributes, as well in the variance in those values, in determining the extent to which modifications to the instructions are made. By considering past differences, the user may avoid making drastic modifications to the instructions based on an outlier difference that is unlikely to repeat- for example, a single large difference based on a defective cartridge during the printing of only one particular product. Further, if there is a standard or known variance in the attributes of printed products, a user might not want to continuously change the instruction set if the difference between an actual product and the desired product falls within this variance. Rather a user can try to hone in on an ideal instruction set which results in equal expected variances in differences between the attributes of actual products and desired products in the positive and negative directions. In light of these considerations, the analysis module may include instructions to determine a variance and only modify the instruction set after a certain number of values have fallen outside this variance. Further, the analysis module may include instructions to ignore single values falling too far outside of an expected variance. Once the instruction set has been modified appropriately, the user can return to step 308 to print new products based on the new instruction set.

After a number of cycles through these steps, step 314 will be reached and there will be little or no differences between the actual product and the desired product. Alternatively, as described above, a user might print a series of products and determine that the group is falling within an expected variance. In either case, the user has created an ideal instruction set and the 3D printer is now creating medical products which are expected to be safe and accurate. Therefore, at step 318, if the user needs additional products of the same type, they can return to step 308 to print additional products based on the ideal instruction set. Once the user no longer wants to create additional products, the method ends at step 320. The user can store the ideal instruction set associated with identifiers related to that particular 3D printer and the particular product that was created so that the instruction set is accessible for future use. Additionally, or alternatively, the controller 152 may automatically store the ideal instruction set in a storage module for later access and use. In some cases, the controller 152 may store the ideal instruction set such that it may be recalled by inputting the identifiers related to the 3D printer and product.

Various methods for measuring product attributes and self-correction are utilized. For example, to measure construction material physical properties, a force sensor is integrated in a print head to measure viscosity, compressibility and/or other viscoelastic properties of extruded material. Measurements are compared against expected measures for the target material. Expected measures and acceptable ranges for viscosity, compressibility and other viscoelastic properties are either (1) experimentally determined and stored in the printer instructions or retrievable from a database storing these property values by target material or (2) computed using mathematical models of physical behavior and measures of the constituent molecules, particularly the active pharmaceutical ingredient, such as molecular weight, dipole moment, and solubility. Acceptable measures of new materials may be determined using experimental measures of similar molecules to perform algorithmic predictive analysis based on key characteristics of the new molecule.

Out-of-range measures are rejected before printing. However, if the material is “new”—it does not have specific measures stored or available in printer instructions or the database—products will be printed and product attributes will be measured to determine suitability and tune printing procedures to accommodate the new material. If desired product attributes are attained during this feedback loop, measures and instructions will be stored for future use with that material.

To determine tablet properties, a computer vision system including at least a camera and laser diode are used to collect images of final products for measurement of key attributes as well as continuous measurement of production attributes, such as rate of material flow onto the build surface. Image analysis algorithms extract product properties, including volume, shape, size and surface aesthetics. These measures may be used as acceptance/rejection criterion or can be compared against an ideal product's properties to adjust printing instructions to improve the next product unit printed. This is repeated for a fixed number of products until ideal attributes are attained, or the starting material is rejected with suggested alterations to achieve these properties.

Measurements of product volume may be computed from readings taken from a motor equipped with an encoder, or alternatively a laser-based range finding system consisting of synchronized camera, laser diode, and motors equipped with encoders. The change in the position and/or encoder reading of the motor responsible for driving material extrusion can be used to compute the volume of material dispensed onto the print stage. Another means of measuring the deposited volume is via a laser-based range finding system. A plane of laser light is projected onto the print stage where the deposited material resides. The laser light plane is then made to sweep over the entirety of the printed volume. The camera records the intersection of the light plane and printed volume as a function of time. Synchronization of the camera, laser diode, and any motors used permit the reconstruction of the printed volume surface from the recorded images. The 2D integral over the reconstructed surface, the height/distance/disparity map, yields the dispensed volume, perhaps with some additional computations or assumptions.

In summary, the present invention is a platform augmenting and tailoring 3D printing (3DP) technology for the unique expectations of drug production and the realities of the pharmacy setting, thus enabling personalized dosage creation. Unlike popular commercial 3DP platforms, the present invention does not use high temperatures (e.g., hot melt extrusion) or UV lasers, either of which can damage active pharmaceutical ingredients (APIs). Our unique room temperature, radiation-free process produces tablets, sublingual oral dissolving tablets, chewable tablets, and flavored gummy dosage forms. The system only requires users to input standard prescription data—e.g., dose and drug—instead of specialized CAD designs required in 3DP. Our hardware design ensures drug material is completely contained in a syringe based cartridge and only touches disposable (the cartridge) or removable and cleanable surfaces (the build tray), preventing cross-contamination. Automated cleaning and controlled airflow have been designed in. Thus, we developed novel systems that determine validity of starting formulations based on measures of unique physical properties, assess accuracy and quality of each final dosage form, and implement the first adaptive pharmaceutical printing process using computer vision. Together, these innovations enable unprecedented continuous QC of personalized dosages and rapid formulation development.

It will be appreciated by those of ordinary skill in the pertinent art that the functions of several elements may, in alternative embodiments, be carried out by fewer elements, or a single element. Similarly, in some embodiments, any functional element may perform fewer, or different, operations than those described with respect to the illustrated embodiment. Also, functional elements (e.g., modules, databases, interfaces, computers, servers and the like) shown as distinct for purposes of illustration may be incorporated within other functional elements in a particular implementation.

While the subject technology has been described with respect to preferred embodiments, those skilled in the art will readily appreciate that various changes and/or modifications can be made to the subject technology without departing from the spirit or scope of the invention as defined by the appended claims.

What is claimed is:

1. A method of creating safe and accurate medical products with a 3D printer comprising:
(a) setting up a 3D printer;
(b) creating a set of printer instructions designed to print a desired final product having at least one desired attribute;
(c) printing a product based on the set of printer instructions;
(d) measuring at least one product attribute to compare with the at least one desired attribute;
(e) determining whether there is a difference between the at least one product attribute and the at least one desired attribute;
(f) modifying the printer instructions based on the difference;
(g) repeating steps (c)-(f) until the at least one product attribute matches the at least one desired attribute; and
(h) printing at least one safe and accurate product where the at least one product attribute matches the at least one desired attribute.

2. The method of claim 1 wherein one of the desired product attributes is a desired product weight and one of the product attributes is a first product weight.

3. The method of claim 2 wherein in step (d) measuring at least one product attribute is accomplished by weighing the product to determine the first product weight.

4. The method of claim 3 wherein the product is weighed on a print stage configured for weight tracking.

5. The method of claim 4 wherein the print stage is configured to weigh products by including, within the print stage, one of the following: force sensing resistors or manometers.

6. The method of claim 3 wherein the at least one desired product attribute is selected depending on a medically recommended dose or a medically recommended volume.

7. The method of claim 1 further comprising the step of:
(i) storing, for future use, a set of printer instructions related to printing the safe and accurate product.

8. The method of claim 1 wherein in step (d) measuring at least one product attribute is accomplished by one of the following techniques: laser based temperature measurements, spectroscopic measurements, or optical measurements.

9. The method of claim 1 wherein the 3D printer is set up in a sterile environment.

10. A method to measure construction material physical properties in 3D medical printing comprises:
integrating a force sensor in a print head and synchronizing with a motor encoder to measure viscosity, and compressibility of extruded material, or alternatively a camera can be used with the above mentioned hardware to measure the aforementioned physical properties by monitoring material flow rate from the print head;
comparing measurements against expected measures for the target material; and
rejecting out-of-range measurements are before printing.

11. The method of claim 10 wherein expected measures and acceptable ranges for viscosity, compressibility and other viscoelastic properties are experimentally determined and stored in printer instructions or retrievable from a database for a specific material

12. The method of claim 10 wherein expected measures and acceptable ranges for viscosity, compressibility and other viscoelastic properties are computed using mathematical models of physical behavior and measures of the constituent molecules, particularly the active pharmaceutical ingredient, such as molecular weight, dipole moment and solubility.

13. The method of claim 10 wherein expected measures and acceptable ranges for viscosity, compressibility and other viscoelastic properties of new construction materials are determined using experimental measures of similar molecules to perform algorithmic predictive analysis based on key characteristics of the new molecule.

14. A method of determining 3D printer product properties comprising:
providing a computer vision system including at least a camera and laser diode to collect images of final products for measurement of key attributes and continuous measurement of production attributes; and
extracting product properties using image analysis to compute measures to compare to the appropriate acceptance/rejection criterion.

15. The method of claim 14 wherein the extracted product properties are used to compare against an ideal product's properties to adjust printing instructions to improve the next product unit printed.

16. The method of claim 14 wherein the production attributes include rate of material flow onto the build surface.
